(19) United States
(12) Patent Application Publication
(10) Pub. No.: US 2026/0325338 A1
(43) Pub. Date: Oct. 22, 2026

(54) NON-INVASIVE GRAVITY TRACTION SYSTEM AND METHOD

(71) Applicant: The Research Institute at Nationwide Children's Hospital, Columbus, OH (US)

(72) Inventors: Allen Kadado, Columbus, OH (US); Jessica Fedor, Columbus, OH (US)

(21) Appl. No.: 19/650,239

(22) Filed: Apr. 16, 2026

Related U.S. Application Data
(60) Provisional application No. 63/789,555, filed on Apr. 16, 2025.

(51) Int. Cl. A61F 5/058; A61F 5/02

(52) U.S. Cl. CPC A61F 5/05816; A61F 5/026

(57) ABSTRACT

A gravity traction assembly for use with a gravity traction system and method of use is provided. The gravity traction assembly includes a vest having a body and side panels, the body of the vest housing a selectively pressurized reservoir, the reservoir having a first pressurization inlet, the first pressurization inlet being accessible through a first inlet of the vest, a shoulder stabilization assembly for coupling to a gravity traction system, and a tubing assembly with at least a portion of the tubing assembly having first and second ends and extending within the body of the vest such that the second end of the portion of the tubing assembly is coupled to the first pressurization inlet of the vest, wherein the reservoir is selectively pressurized through the second end of the portion of the tubing assembly to create a treatment pressure gradient.

### CROSS REFERENCES TO RELATED APPLICATIONS

The following application claims priority under 35 U.S.C. § 119 (e) to U.S. Provisional Patent Application Ser. No. 63/789,555 filed Apr. 16, 2025 entitled NON-INVASIVE GRAVITY TRACTION SYSTEM AND METHOD. The above-identified application is incorporated herein by reference in its entirety for all purposes.

### TECHNICAL FIELD

The present disclosure generally relates to a non-invasive gravity traction system for at-home application and use, and more particularly to a gravity traction assembly having a vest with a selectively adjustable reservoir for spinal correction.

### BACKGROUND

Traditional halo gravity traction is used to treat severe spinal deformities by gradually lengthening and straightening the spinal column of a user in preparation for spinal fusion surgery. The gradual correction allows the spine to be manipulated in a manner that is safe for the spinal cord. The process typically begins with the surgical insertion of pins to anterior and posterior locations on a patient's head. A halo ring is affixed through these pins, and the halo is then attached to a traction device that includes a pulley system. A traditional halo gravity traction assembly is shown in FIG. 1.

In some patient populations, including pediatric patients, for whom spinal surgery with instrumentation would present high risks of complications including infections, revisions needed, high anesthesia risk, iatrogenic stiffness, and/or multiple reoperations, long-term use of halo gravity traction is needed. In a treatment plan for some patient populations which utilizes long-term halo gravity traction, a patient stays in a supervised hospital setting for approximately two-six weeks before discharge to home with a halo gravity traction system for use until the patient is medically fit to receive a spinal instrumentation operation. In other cases, some patient populations are already deemed medically fit and able to undergo spinal instrumentation procedures, and a treatment plan for these patient populations includes a short-term hospital stay, two to four weeks at home using halo gravity traction, and an eventual return to the hospital setting for a spinal instrumentation operation.

In other cases, some patient populations, including pediatric patients, are unable or do not desire to undergo an anesthesia event for halo application. For these patient populations, a gravity traction device without a surgically placed halo would be advantageous.

### SUMMARY

In one aspect, the present disclosure includes a gravity traction assembly for use with a gravity traction system for reducing spinal deformity of a user. The gravity traction assembly includes a vest having a body and side panels, the body of the vest housing a selectively pressurized reservoir, the reservoir having a first pressurization inlet, the first pressurization inlet being accessible through a first inlet of the vest, a closure system including a plurality of fasteners, a shoulder stabilization assembly having a support bar extending from the body of the vest, the support bar for coupling to a gravity traction frame assembly; and a tubing assembly extending across the body of the vest with at least a portion of the tubing assembly having first and second ends and extending within the body of the vest such that the second end of the portion of the tubing assembly is coupled to the first pressurization inlet of the vest, and the first end being removably couplable to an external pressurization element, wherein the reservoir is selectively pressurized through the second end of the portion of the tubing assembly to create a treatment pressure gradient for the gravity traction assembly.

In another aspect, the present disclosure includes a method of manufacturing a gravity traction assembly for use with a gravity traction system for reducing spinal deformity of a user. The method includes the steps of providing a vest having a body and side panels, the body of the vest including a cavity for housing a selectively pressurized reservoir, forming a first inlet within the vest which corresponds with a first pressurization inlet of a selectively pressurized reservoir, attaching a closure system including a plurality of fasteners coupled to the vest, forming a first coupling inlet for supporting a portion of a pressurization tubing assembly within the vest, and attaching first and second adjustable shoulder straps to the vest, wherein each of the first and second shoulder straps includes a looped portion housing first and second strap couplings corresponding with the first and second shoulder straps.

In yet another aspect, the present disclosure includes a method of using a gravity traction assembly for use with a gravity traction system for reducing spinal deformity of a user, The method includes the steps of providing a vest having a body and first and second side panels, the vest housing a selectively pressurized reservoir having a first pressurization inlet corresponding with a first inlet of said vest, the vest further including first and second straps having a looped portion supporting first and second strap couplings, the vest further comprising a tubing assembly having first and second ends extending across the body of the vest with at least a portion of the tubing assembly extending within the body of the vest such that the second end of the portion of the tubing assembly extending within the body of the vest is removably couplable to the first pressurization inlet of the vest, and the first end of the tubing assembly is removably couplable to an external pressurization element, coupling the second end of the tubing assembly to the first pressurization inlet of the vest; placing the vest on a patient's body, securing the vest from an open position to a closed position with a closure assembly secured to the vest, and coupling a support bar attachment to the first and second strap couplings of the vest.

### BRIEF DESCRIPTION OF THE DRAWINGS

The foregoing and other features and advantages of the present disclosure will become apparent to one skilled in the art to which the present disclosure relates upon consideration of the following description of the disclosure with reference to the accompanying drawings, wherein like reference numerals refer to like parts unless described otherwise throughout the drawings and in which:

FIG. 1 is a prior art halo gravity traction system;

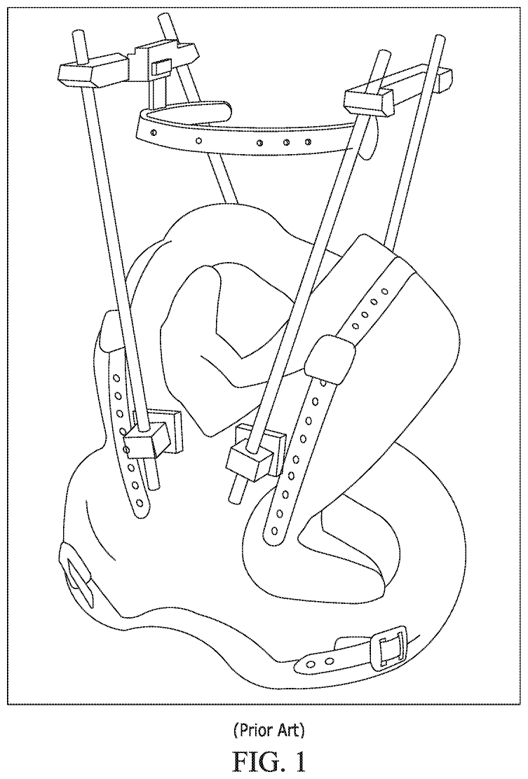

FIG. 2 illustrates a rear interior view of a gravity traction assembly in an open position in accordance with one example embodiment of the present disclosure;

FIG. 3 illustrates an optional head assembly in accordance with another example embodiment of the present disclosure;

FIG. 4 illustrates another rear interior view of a gravity traction assembly in an open position in accordance with another example embodiment of the present disclosure;

FIG. 5 illustrates a side exterior view of a gravity traction vest in accordance with another example embodiment of the present disclosure;

FIG. 6 illustrates a rear view of a gravity traction assembly in accordance with another example embodiment of the present disclosure;

FIG. 7 illustrates a front exterior view of a gravity traction assembly position in accordance with another example embodiment of the present disclosure;

FIG. 8 illustrates a connection assembly in accordance with another example embodiment of the present disclosure;

FIG. 9 illustrates another rear interior view of a gravity traction assembly in accordance with another example embodiment of the present disclosure;

FIG. 10 illustrates a rear view of a gravity traction vest including an attachment line in accordance with another example embodiment of the present disclosure;

FIG. 11 illustrates shoulder pads in accordance with another example embodiment of the present disclosure;

FIG. 12 illustrates an exterior view of a gravity traction assembly in an open position and including an attachment assembly in accordance with another example embodiment of the present disclosure;

FIG. 13 illustrates an exterior view of a gravity traction assembly in an open position and including an attachment assembly in accordance with another example embodiment of the present disclosure;

FIG. 14 illustrates a rear interior view of a gravity traction assembly in an open position and including an attachment assembly in accordance with another example embodiment of the present disclosure; and

FIG. 15 is a method of use for a gravity traction assembly in accordance with another example embodiment of the present disclosure.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present disclosure.

The apparatus and method components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

### DETAILED DESCRIPTION

Referring now to the figures generally wherein like numbered features shown therein refer to like elements throughout unless otherwise noted. The present disclosure generally relates to a non-invasive gravity traction system for at-home application and use, and more particularly to a gravity traction assembly having a vest with a selectively adjustable reservoir for spinal correction.

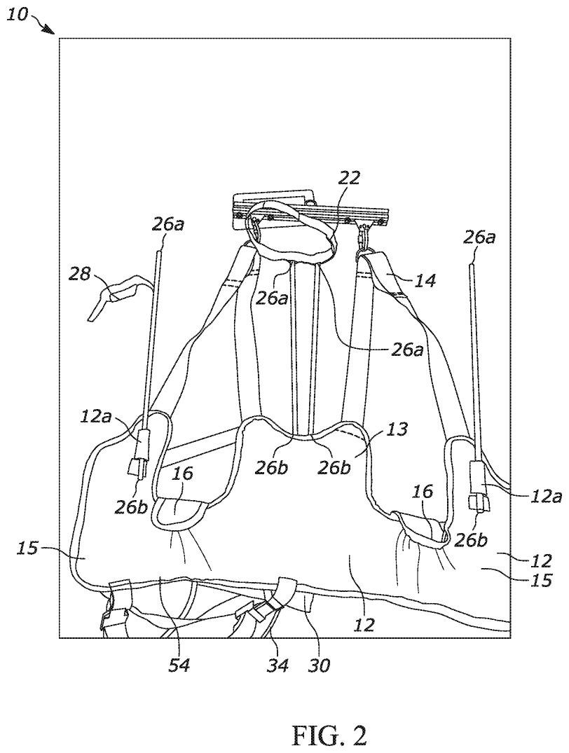

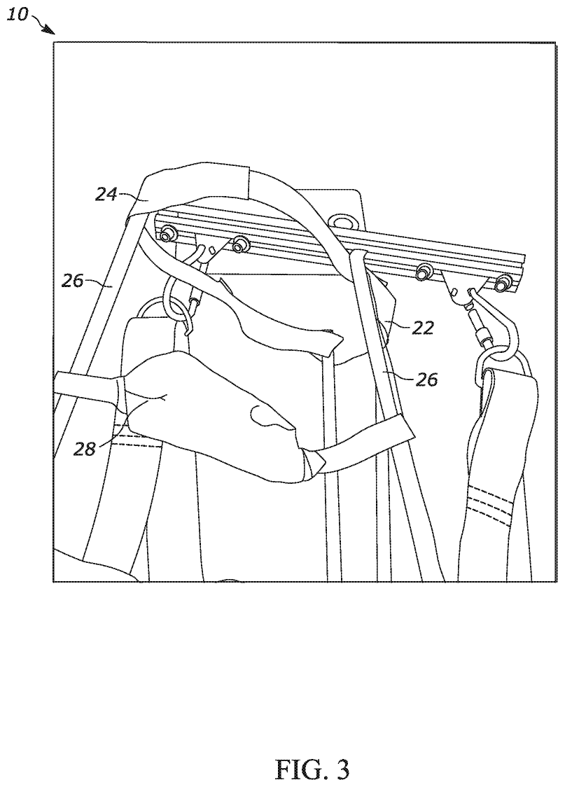

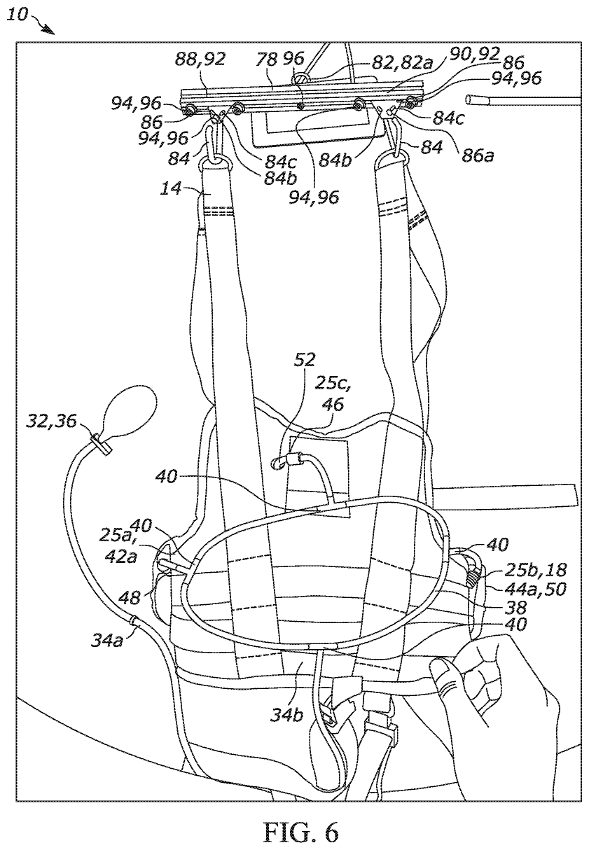

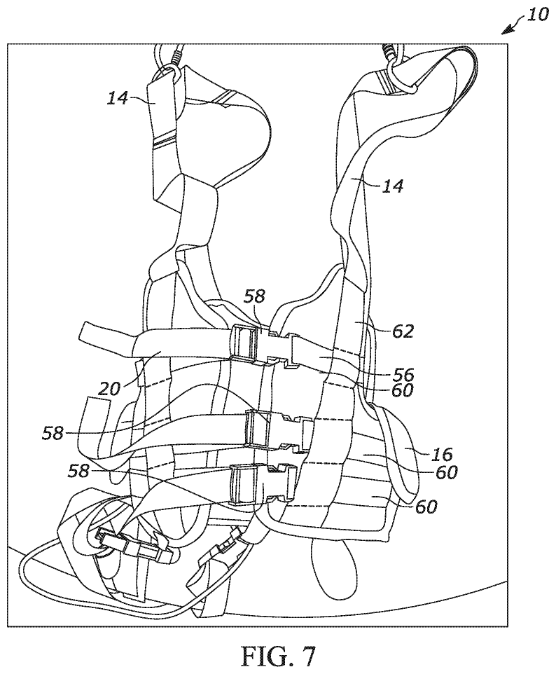

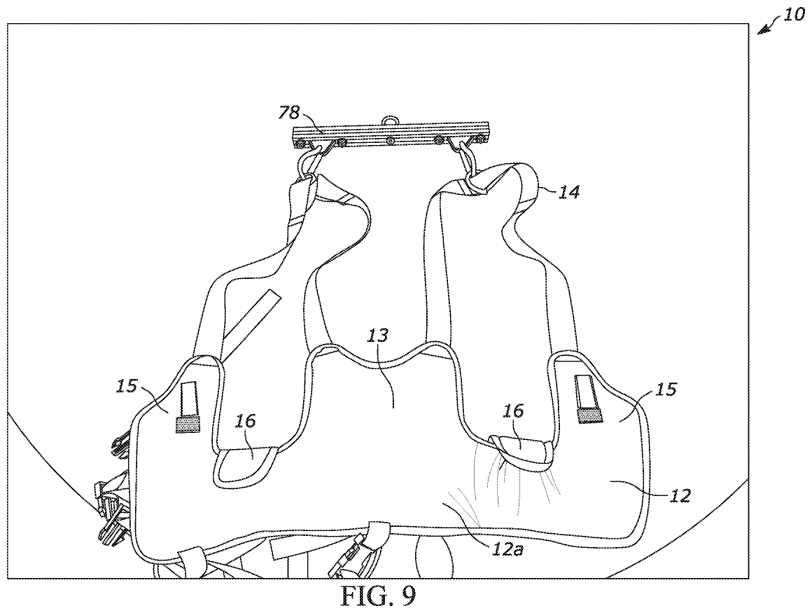

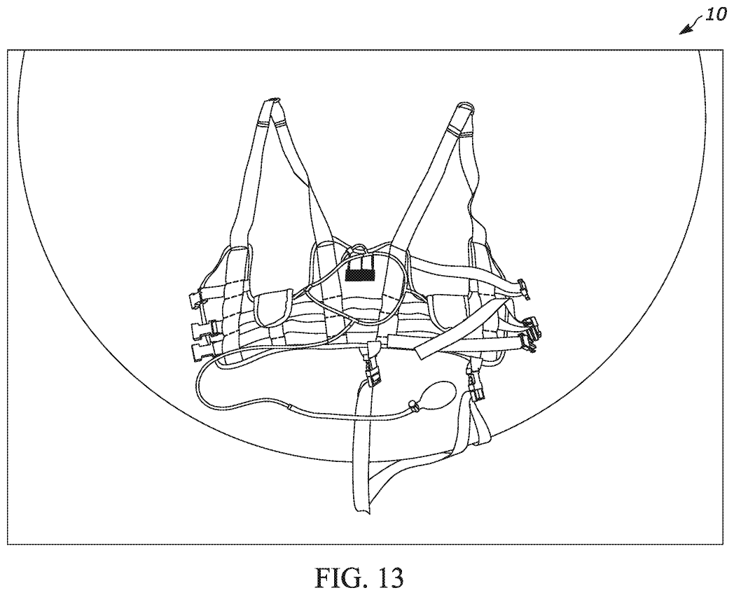

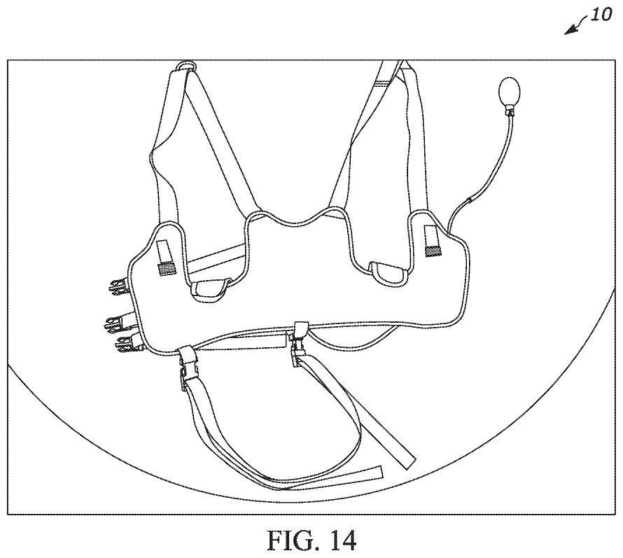

Illustrated in FIGS. 2-10 and 13-14 is a gravity traction assembly 10 in accordance with one example embodiment of the present disclosure. The gravity traction assembly 10 includes a traction vest 12, shoulder support system 14, underarm supports 16, tubing assembly or manifold 18 (see FIG. 6), and closure system 20. Advantageously, the gravity traction assembly 10 can be worn with or without optional head assembly 22, as shown in FIG. 3.

Optional head assembly 22, as shown in FIG. 3, advantageously allows users (without head control) to use the gravity traction assembly 10. The optional head assembly 22 includes an adjustable head strap 24, side supports 26, and chin strap 28 extending from one side support 26. The adjustable head strap 24, in one example embodiment of the present disclosure, is constructed from polyester or an equivalent fabric with hook and loop fasteners sewn to the head strap 24 such that a user can easily adjust and secure the positioning of the optional head assembly 22. The side supports 26 extend orthogonally from the head strap 24 and are stiff in nature, being constructed from wire, metal, and/or a polymeric material covered with fabric in one example embodiment of the present disclosure. The side supports 26 have first 26a and second 26b ends, with the first 26a ends mating with the hook and loop fasteners of the adjustable head strap 24 when the head assembly 22 is in an assembled configuration. The second ends 26b of the side supports 26 are coupled with the traction vest 12. In this example embodiment, the traction vest 12 includes coupling loops 12a which frictionally secure the second ends 26b of the side supports 26 to the traction vest 12.

When the optional head assembly 22 and remaining parts of the gravity traction assembly 10 are coupled with a traditional halo gravity traction frame (see, for example the system described and illustrated in International Patent Application Serial Number PCT/US2023/030952 that was published on Feb. 29, 2024 under publication number WO 2024/044267 A1 entitled DYNAMIC HALO GRAVITY TRACTION SYSTEM AND METHOD, which is incorporated herein by reference in its entirety), a user of the gravity traction assembly 10 is advantageously held upright and gravity allows for the positioning of the remainder of the body distally. While, in this example embodiment, the gravity traction assembly 10 with a head assembly 22 is used for patients having thoracolumbar and/or lumbar scoliosis in hypotonic neuromuscular conditions, use with other types of scoliosis is also contemplated. In one example embodiment, the gravity traction assembly 10 is compatible with a standing support sling manufactured by Savaria. In another example embodiment, the gravity traction assembly 10 is compatible with a support frame manufactured by Zing, such as the PK5010 Zing Portable.

As shown in FIG. 2, the traction vest 12 has a body 13 and side panels 15. The body 13 of traction vest 12 houses a reservoir 25 within which pressurization can be adjusted to assist in correcting spinal curvature of a user of the gravity traction system 10 through a tubing assembly 18 coupled to the reservoir 25. In one example embodiment of the present disclosure, this pressurization is achieved through injected compressed air into the reservoir 25. In this example embodiment of the present disclosure, a tubing assembly 18 is used to circulate air to the reservoir 25. The tubing assembly 18 is pneumatic tubing in this example embodiment and can be constructed from a variety of flexible polyvinyl or equivalent materials. Advantageously, the pressure to which the reservoir 25 is inflated can be adjusted based on patient comfort, size of a patient, and/or overall treatment time. In one example embodiment of the present disclosure, the reservoir 25 is pressurized to a range of approximately 2 to 35 psi to create a set treatment pressure 27.

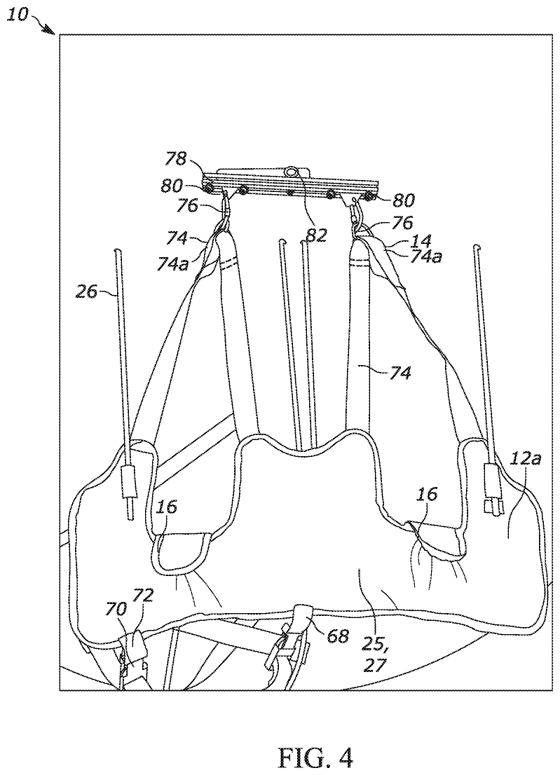

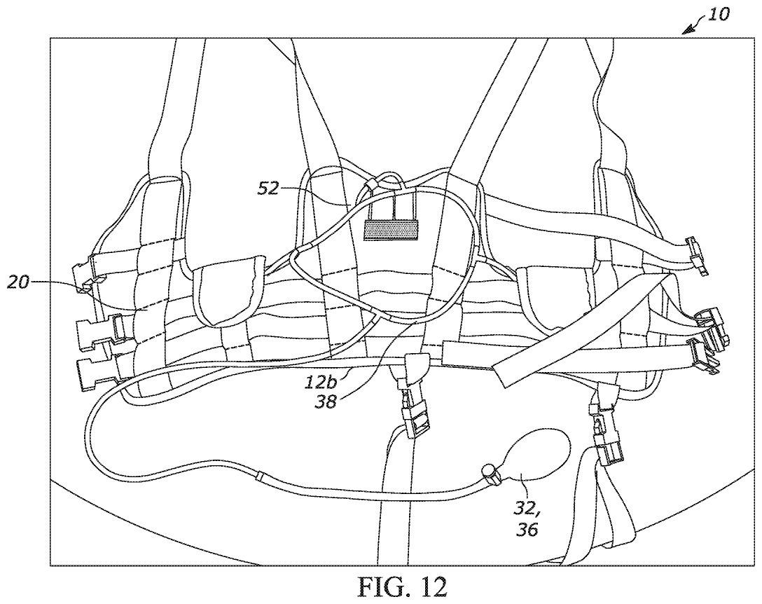

To pressurize the reservoir, as shown in FIG. 12, an external line 34 of the tubing assembly 18 extends from reservoir 25 (see FIG. 4) out of the vest 12. An external pressurization element 32, such as a bulb pump (see FIG. 6) in this example embodiment, is coupled to a first end 34a of external line 34 such that compressed air travels through external line 34 to its second end 34b, which is coupled with additional portions of the tubing assembly 18 as described below to pressurize the reservoir 25. A pressure gauge 36 (see FIG. 6) can be coupled to the external pressurization element 32 to ensure that set treatment pressure 27 is achieved for the gravity traction assembly 10.

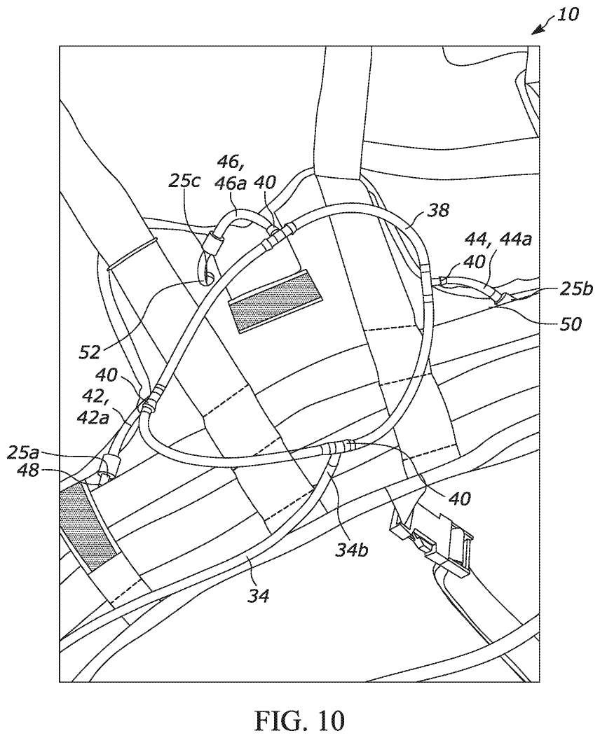

The tubing assembly 18 also includes a substantially circular portion 38 (see FIG. 6) transversely coupled to one of a plurality of connectors 40 coupled to the second end 34b of external line 34. In the example embodiment shown in FIG. 10, the tubing assembly 18 utilizes a plurality of connectors 40 that are T-shaped. The circular portion 38 of the tubing 18 extends across an exterior portion 12b of the body 13 of the vest 12 opposite the user's back and includes three additional connectors 40 coupled to the length of the circular portion 38. These connectors 40 are coupled to first ends 42a, 44a of first 42 and second 44 pressurization lines, respectively, which are located near the underarm supports 16 of the vest 12 and have second ends 42b, 44b which are coupled to the reservoir 25 at ports 25a, 25b. Additionally, a third pressurization line 46 is included, with its first end 46a being coupled to circular portion 38 and second end extending into the vest 12 and coupled with reservoir 25 at port 25c. Advantageously, the ports 25a-25c are adjustable to allow for the reservoir 25 to retain pressurization or to lessen pressure if needed.

The vest 12 includes first 48, second 50, and third 52 inlets formed within which allow passage of the lines 42, 44, and 46 into the body 13 of the vest 12 where the reservoir 25 is housed. Advantageously, the delivery of pressurized air to the reservoir 25 through multiple ports and through circular portion 38 allows for precision of pressurization and equalization of pressure throughout the vest 25.

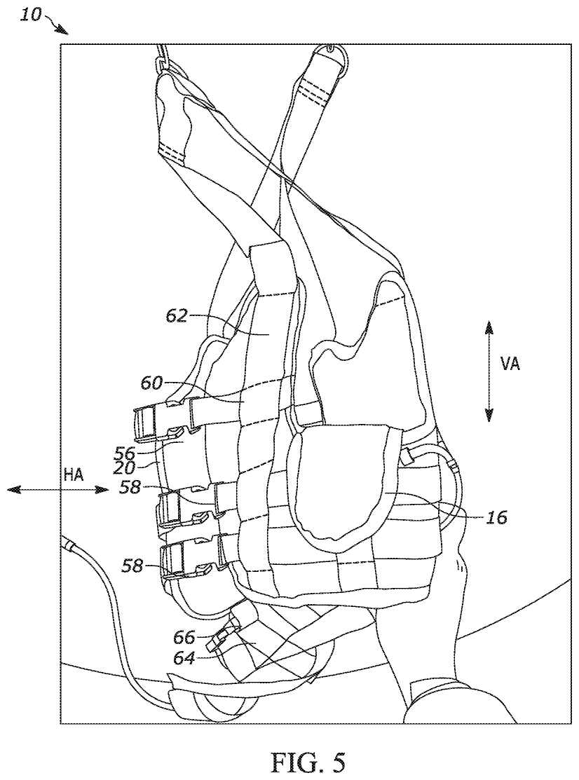

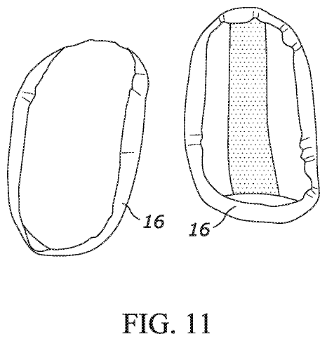

The vest 12 also includes a pair of removable underarm supports 16 (see FIG. 11) for user comfort when wearing the gravity traction assembly 10. In this example embodiment of the present disclosure, the underarm supports 16 are constructed from a padded fabric. The vest 12 can be secured from an open position 54 to a closed position 56 by a network of fasteners 58 located on the vest panels 15 (see FIG. 5). In this example embodiment of the present disclosure, the fasteners 58 extend from a plurality of straps 60 extending across a horizontal axis HA woven through additional straps 62 coupled to the body 13 of the vest 12 along a vertical axis VA. In one example embodiment, the gravity traction assembly 10 includes a groin strap 64 which extends from a fastener 66 located on the rear edge 68 of the vest to a fastener 70 on a front edge 72 of the vest to prevent a user from slipping out of the gravity traction assembly 10.

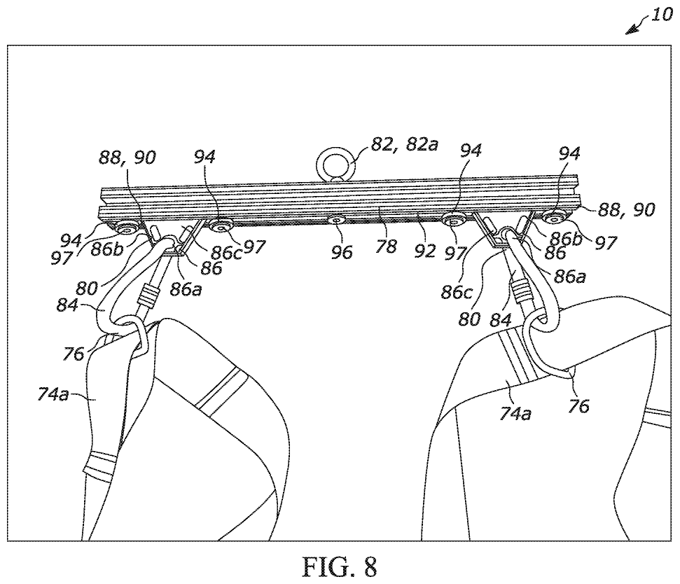

As shown in FIGS. 4 and 8, the shoulder support system 14 of the gravity traction assembly 10 includes shoulder straps 74, strap couplings 76, support bar 78, support bar attachments 80, and support bar coupling 82. The shoulder straps 74 are made of polyester or an equivalent woven fabric and are adjustable based on a user height and/or desired tension level of a user in one example embodiment of the present disclosure. The shoulder straps 74 include a looped portion 74a which houses a strap coupling 76 on each strap 74. In one example embodiment of the present disclosure, the strap couplings 76 are stainless steel D-rings. One of ordinary skill in the art, after reviewing the specification and drawings of the present disclosure, would understand that a variety of shapes and metal/non-metal materials are contemplated for the strap couplings 76 and are included within the spirit and scope of the present disclosure.

In one example embodiment of the present disclosure, support bar attachments 80 include a pair of clips 84 hung from coupling members 86. In this example embodiment, the clips 84 are carabiner clips. As can be seen in FIG. 8, the coupling members 86 include first inlets 86a for receiving the clips 84 and include reliefs 86b, 86c so that the coupling members 86 can effectively support the weight of a patient using the gravity traction system 10. In this example embodiment, the coupling members 86 have a polygonal shape. The coupling members 86 include first 88 and second 90 flanges (not shown) which extend transversely from the coupling members 86 and are coupled to an interior track 92 created within support bar 78. The flanges 88, 90 are coupled in place through threaded support channels 94 along the interior track 92 of the support bar 78 with fasteners 97 that have corresponding threaded bodies such as screws.

As also shown in FIGS. 4 and 8, the support bar 78 also includes a threaded main channel 96 which receives a threaded body 82a of support bar coupling 82. The support bar coupling 82, a hook in this example embodiment, is composed of stainless steel, iron, or an equivalent metal or non-metal material with equivalent strength and durability, and allows the traction assembly 10 to be coupled with an external frame assembly (not shown), as is used in a traditional halo gravity traction assembly.

Advantageously, the gravity traction assembly 10 of the present disclosure allows users that cannot tolerate a surgical procedure and/or anesthetic event that would be involved with placing a traditional halo for gravity traction to access the benefits of traditional gravity traction, which allows for correction of spinal curvature prior to spinal fusion surgery. Moreover, the gravity traction assembly 10 without a traditional halo allows for flexibility of use for users and their caretakers, allowing for at-home use of the system 10 and avoiding a lengthy hospital stay.

Additionally, the gravity traction assembly 10 shown and described in the present disclosure is non-invasive in comparison to traditional halo gravity traction assemblies, and circumvents complications associated with traditional halo application, which include complications with anesthesia, pin site infection, loosening of the halo, and/or other issues related to traditional pins in relation to a user's cranium in relation to a traditional halo gravity traction assembly. Advantageously, the gravity traction assembly 10 provides medicinal support, for example straightening skeletal and muscular systems while the system is suspended by the support bar 78 to a traditional suspension frame through the vest 12, reservoir 25, and/or manifold 18 that allows for increased pressure and holding forces during use. The parts of the gravity traction assembly 10 described herein can be used to modify or adjust currently existing vests, straps, hoyer lifts, standing frames, etc. currently being used in connection with non-invasive gravity traction assemblies for spinal deformity.

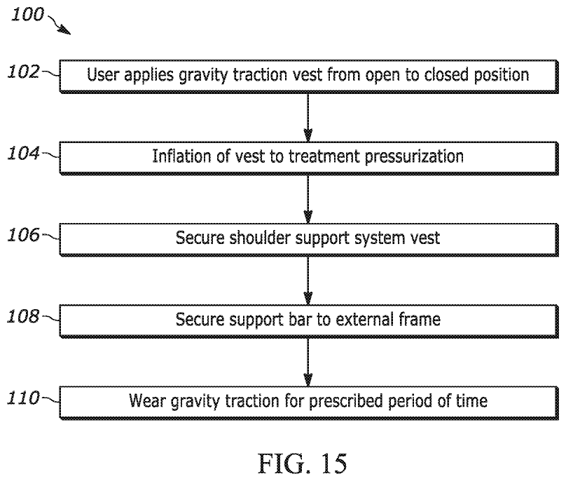

As shown in FIG. 15, a method of operation 100 for the gravity traction assembly 10 will now be described. At method step 102, a traction vest 12 having a shoulder strap assembly 14 and a reservoir 25 is secured to a body of a patient from an open position 54 to a closed position 56 with closure assembly 20. At step 104, the traction vest 12 housing the reservoir 25 is inflated by an external pump 32 through the external line 34 and travels through tubing assembly 18 to the reservoir 25 until it reaches a treatment pressurization 27. At step 106, a shoulder support system 14 having straps 74 coupled to strap couplings 76 is coupled to a support bar 78 by support bar attachments 80. At step 108, the support bar 78 coupling 82 is coupled to an external gravity traction frame assembly (not shown). At step 110, the patient wears the gravity traction assembly 10 for a prescribed length of time before removal.

In the foregoing specification, specific embodiments have been described. However, one of ordinary skill in the art appreciates that various modifications and changes can be made without departing from the scope of the disclosure as set forth in the claims below. Accordingly, the specification and figures are to be regarded in an illustrative rather than a restrictive sense, and all such modifications are intended to be included within the scope of present teachings.

The benefits, advantages, solutions to problems, and any element(s) that may cause any benefit, advantage, or solution to occur or become more pronounced are not to be construed as a critical, required, or essential features or elements of any or all the claims. The disclosure is defined solely by the appended claims including any amendments made during the pendency of this application and all equivalents of those claims as issued.

Moreover, in this document, relational terms such as first and second, top and bottom, and the like may be used solely to distinguish one entity or action from another entity or action without necessarily requiring or implying any actual such relationship or order between such entities or actions. The terms “comprises,” “comprising,” “has”, “having,” “includes”, “including,” “contains”, “containing” or any other variation thereof, are intended to cover a non-exclusive inclusion, such that a process, method, article, or apparatus that comprises, has, includes, contains a list of elements does not include only those elements but may include other elements not expressly listed or inherent to such process, method, article, or apparatus. An element proceeded by “comprises . . . a”, “has . . . a”, “includes . . . a”, “contains . . . a” does not, without more constraints, preclude the existence of additional identical elements in the process, method, article, or apparatus that comprises, has, includes, contains the element. The terms “a” and “an” are defined as one or more unless explicitly stated otherwise herein. The terms “substantially”, “essentially”, “approximately”, “about” or any other version thereof, are defined as being close to as understood by one of ordinary skill in the art. In one non-limiting embodiment the terms are defined to be within for example 10%, in another possible embodiment within 5%, in another possible embodiment within 1%, and in another possible embodiment within 0.5%.

The term “coupled” as used herein is defined as connected or in contact either temporarily or permanently, although not necessarily directly and not necessarily mechanically. A device or structure that is “configured” in a certain way is configured in at least that way but may also be configured in ways that are not listed. The term “integral” as used herein unless defined otherwise means configured in such a way that separation would require destruction to the parts or the assembly of the parts.

It should be appreciated by those of ordinary skill in the art after having the opportunity of reviewing the drawings and/or specification of the present disclosure that it may include one or more embodiments, e.g., E₁, E₂, . . . Eₙ and that each embodiment E may have multiple parts A₁, B₁, C₁. . . . Zₙ that (without further description) could be combined with other embodiments Eₙ, embodiment parts e.g. A₁, C₁, or lack of parts originally associated with one or all embodiments Eₙ, or any combination of parts and/or embodiments thereof. It should further be appreciated that an embodiment Eₙ may include only one part e.g. A₁ or a lesser number of parts e.g. B₁, C₁ of any embodiment or combination of embodiments that was described or shown in the specification and/or drawings, respectively in ways not enumerated or illustrated.

To the extent that the materials for any of the foregoing embodiments or components thereof are not specified, it is to be appreciated that suitable materials would be known by one of ordinary skill in the art for the intended purposes after having the benefit of reviewing the subject disclosure and accompanying drawings.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

What is claimed is:

1. A gravity traction assembly for use with a gravity traction system for reducing spinal deformity of a user, said gravity traction assembly comprising:
a vest having a body and side panels, said body of said vest housing a reservoir that can be selectively pressurized, said reservoir having a first pressurization inlet, said first pressurization inlet being accessible through a first inlet of said vest;
a closure system comprising a plurality of fasteners;
first and second shoulder straps having first and second strap couplings;
a shoulder stabilization assembly removably couplable to said first and second strap couplings, said shoulder stabilization assembly having a support bar extending from said body of said vest, said support bar for coupling to a gravity traction frame assembly; and
a tubing assembly having first and second ends extending across the body of said vest with at least a portion of said tubing assembly and extending within the body of said vest such that the second end of said portion of said tubing assembly is coupled to said first pressurization inlet of said vest, and said first end is removably couplable to an external pressurization element;
wherein said reservoir can be selectively pressurized through said second end of said portion of said tubing assembly to create a treatment pressure gradient for said gravity traction assembly.

2. The gravity traction assembly of claim 1 further comprising a removeable head assembly removably coupled to said vest by at least one side support.

3. The gravity traction assembly of claim 2, wherein said head assembly includes an adjustable head strap.

4. The gravity traction assembly of claim 1, wherein said treatment pressure gradient ranges between 2 and 35 psi.

5. The gravity traction assembly of claim 1, wherein said first and second strap couplings are removably coupled to said shoulder stabilization assembly by first and second clips supported by first and second coupling members extending from said shoulder stabilization assembly.

6. The gravity traction assembly of claim 1 further comprising underarm supports removably couplable with said vest.

7. The gravity traction assembly of claim 1, wherein said external pressurization element is a bulb pump.

8. The gravity traction assembly of claim 1, wherein said reservoir further comprises a second pressurization inlet.

9. A method of manufacturing a gravity traction assembly for use with a gravity traction system for reducing spinal deformity of a user, the method comprising the steps of:
providing a vest having a body and side panels, said body of said vest including a cavity for housing a selectively pressurized reservoir;
forming a first inlet within said vest which corresponds with a first pressurization inlet of a selectively pressurized reservoir;
attaching a closure system comprising a plurality of fasteners coupled to said vest;
forming a first coupling inlet for supporting a portion of a pressurization tubing assembly within said vest; and
attaching first and second adjustable shoulder straps to said vest, wherein each of said first and second shoulder straps includes a looped portion housing first and second strap couplings corresponding with said first and second shoulder straps.

10. The method of claim 9 further comprising the step of providing a removeable head assembly removably coupled to said vest, said head assembly comprising an adjustable head strap and an adjustable chin strap extending from at least one side support.

11. The method of claim 9 further comprising the step of coupling a pair of removeable underarm supports to said vest.

12. The method of claim 9 further comprising the step of attaching a groin strap to a front edge and a rear edge of said vest, said groin strap being removably couplable by a mating closure.

13. The method of claim 9 further comprising the step of inserting a selectively pressurized reservoir within said cavity of said vest.

14. The method of claim 9 further comprising the step of forming a second inlet within said vest which corresponds with a said second pressurization inlet of a selectively pressurized reservoir.

15. A method of using a gravity traction assembly for use with a gravity traction system for reducing spinal deformity of a user, the method comprising the steps of:
providing a vest having a body and first and second side panels, the vest housing a selectively pressurized reservoir having a first pressurization inlet corresponding with a first inlet of said vest, the vest further including first and second straps having a looped portion supporting first and second strap couplings, the vest further comprising a tubing assembly having first and second ends extending across the body of the vest with at least a portion of the tubing assembly extending within the body of the vest such that the second end of the portion of the tubing assembly extending within the body of the vest is removably couplable to the first pressurization inlet of the vest, and the first end of the tubing assembly is removably couplable to an external pressurization element;
coupling the second end of the tubing assembly to the first pressurization inlet of the vest;
placing the vest on a patient's body;
securing the vest from an open position to a closed position with a closure assembly secured to the vest; and
coupling a support bar attachment to the first and second strap couplings of the vest.

16. The method of claim 15 further comprising the step of coupling a removably couplable head assembly to the vest.

17. The method of claim 15 further comprising the step of coupling the first end of the tubing assembly to an external pressurization element.

18. The method of claim 17 further comprising the step of pressurizing the reservoir of the vest through the external pressurization element coupled to the first end of the tubing assembly to a treatment pressurization ranging from 2 to 35 psi.

19. The method of claim 15 further comprising the step of coupling removeable underarm supports to the vest.

20. The method of claim 18 further comprising the step of coupling a pressure gauge to the external pressurization element.
